(19) United States
(12) Patent Application Publication
(10) Pub. No.: US 2015/0328316 A1
(43) Pub. Date: Nov. 19, 2015

(54) Magnesium hydroxide carbonate as excipient in pharmaceutical preparations having improved release of active ingredient

(71) Applicant: MERCK PATENT GMBH, Darmstadt (DE)

(72) Inventors: Guenter MODDELMOG, Reinheim (DE); Roberto OGNIBENE, Darmstadt (DE); Thorsten WEDEL, Stockstadt/Rhein (DE); Dieter LUBDA, Bensheim (DE)

(73) Assignee: MERCK PATENT GMBH, Darmstadt (DE)

(21) Appl. No.: 14/653,507

(22) PCT Filed: Nov. 22, 2013
(86) PCT No.: PCT/EP2013/003537

§ 371 Date: Jun. 18, 2015

(30) Foreign Application Priority Data
Dec. 21, 2012 (EP) 12008593.1
Feb. 5, 2013 (EP) 13000573.9

(51) Int. Cl. A61K 47/02; A61K 31/216

(52) U.S. Cl. CPC A61K 47/02; A61K 31/216

(57) ABSTRACT

The present invention relates to pharmaceutical formulations of active ingredients which have low solubility in aqueous solutions, having improved release of active ingredient, and to a process for the preparation thereof. In particular, these are pharmaceutical preparations in which magnesium hydroxide carbonate serves as excipient.

The present invention relates to pharmaceutical formulations of active ingredients which have low solubility in aqueous solutions, having improved release of active ingredient, and to a process for the preparation thereof. In particular, these are pharmaceutical preparations in which magnesium hydroxide carbonate serves as excipient.

### PRIOR ART

Active pharmaceutical ingredients (APIs) for use in pharmaceutical preparations must have processing properties which are usable for pharmaceutical practice in order that an administration form which is suitable for patients can be formulated at all. On the other hand, the active ingredients must also be released again from these formulations in the body in order to develop their physiological action. In particular, low-solubility active ingredients cause problems here, since they are insoluble per se and also cannot be dissolved out of their formulations sufficiently quickly, if at all, and are thus not absorbed sufficiently in the body in order to serve their medical purpose. Active-ingredient release of problem medicaments having low solubility can be accelerated by chemical or physical manipulations on the API, through the use of an additive which improves the dissolution rate or by skilful choice of a medicament excipient.

### OBJECT OF THE PRESENT INVENTION

The object of the present invention is therefore to provide formulations and processes by means of which active ingredients having low solubility are converted into a form in which they can be made more easily bioavailable after administration to the patient. In particular, the object consists in forming a stable bond between a physiologically tolerated, porous magnesium hydroxide carbonate in a form by means of which the solubility rate of the low-solubility active ingredient is improved and its release from medicament forms is accelerated, so that the prerequisites for improved absorption and bioavailability are provided.

### BRIEF DESCRIPTION OF THE INVENTION

The present invention relates, in particular, to formulations having accelerated active-ingredient release which comprise porous magnesium hydroxide carbonate having a large surface area as excipient material and at least one active ingredient. The active ingredients present in the formulation according to the invention are preferably at least one pharmaceutical active ingredient. Corresponding pharmaceutical formulations according to the invention having better bioavailability comprise at least one low-solubility active ingredient from BCS classes II and IV.

Surprisingly, the improved release of the low-solubility active ingredient(s) is achieved by applying it or them to the porous magnesium hydroxide carbonate as excipient material by adsorption from a solution. In this connection, the better active-ingredient release is achieved in a satisfactory manner if the excipient material employed is magnesium hydroxide carbonate having a BET surface area of at least 20 m²/g, preferably at least 30 m²/g and particularly preferably at least 40 m²/g, and corresponding pharmaceutical formulations are prepared therefrom. Particularly good properties in this connection are exhibited by formulations in which porous magnesium hydroxide carbonate having a BET pore volume of at least 0.1 cm³/g is present as excipient material.

These formulations according to the invention can be prepared in accordance with the invention in a simple process in which, in a first step, the active ingredient(s) is (are) dissolved in a suitable solvent or solvent mixture in which the porous magnesium hydroxide carbonate is insoluble. In the following process step, the resultant solution is mixed intimately with the porous magnesium hydroxide carbonate, and the solvent or solvent mixture is subsequently removed again after the mixing. The removal of the solvent or solvent mixture can be carried out with the aid of a wide variety of convection and contact drying methods. A particularly good bond between the magnesium hydroxide carbonate and the active ingredient(s) which has (have) low solubility in aqueous solutions is obtained if the method is carried out using fluidised-bed drying, and the solution comprising active ingredient is brought into intimate contact at low temperature by spraying onto a porous magnesium hydroxide carbonate which is initially introduced in a fluidised bed, and dried. However, it has also been shown that a good bond between the excipient and the active ingredient(s) which has (have) low solubility in aqueous solutions is likewise obtained if the porous magnesium hydroxide carbonate is suspended in the solution comprising active ingredient and is subsequently dried in a spray- or freeze-drying process.

### DETAILED DESCRIPTION

Active pharmaceutical ingredients which have low solubility in aqueous solutions generally also have poor bioavailability. (Active pharmaceutical ingredients are referred to below as APIs). The primary aim in the case of problem medicaments of this type is therefore to increase the dissolution rate in order to release and make available for absorption a sufficient amount of active ingredient in the body. Only thus can a sufficiently high active-ingredient concentration be achieved at the desired site of action in the body.

The chemical and physical manipulations on the API which are frequently used in order to increase the dissolution rate often cause toxicological and technical problems, as does the use of solubilising additives, since additives of this type frequently exhibit negative physiological interactions with body tissues.

These problems can be solved by adsorbing the problem medicament onto a physiologically acceptable porous excipient. Magnesium hydroxide carbonates having a particularly large BET surface area and a high pore volume are particularly suitable for this purpose since they are regarded as substantially physiologically safe.

The large surface area and the high pore volume of the magnesium hydroxide carbonate used enable the problem medicament to be uniformly distributed on this surface and in the pores. At the same time, the available contact area for a possible interaction with the release medium or the physiological liquids is thereby increased. This can be carried out by preparing a simple physical mixture of the crystalline API with the specific magnesium hydroxide carbonate. However, for a good bond between the magnesium hydroxide carbonate excipient and the crystalline APIs, it is important for the latter preferably to be initially introduced in the form of particularly small particles, so that a corresponding physical interaction enables the formation of a bond between excipient and API.

Experiments have now shown that a better bond between the magnesium hydroxide carbonate excipient and the low-solubility medicaments can be produced if the latter are dissolved in a suitable solvent and this solution is then brought into intimate contact with the porous magnesium hydroxide carbonate, so that the surface and pores of the excipient can be covered or filled with the active-ingredient solution. The solvent is subsequently removed. In this way, a medicament/magnesium hydroxide carbonate conglomerate can be formed by drying-off of the solvent.

The increase in the contactable medicament surface area which occurred in the process and, where appropriate, also the amorphous structures forming significantly increase the release rate of APIs, in particular in the case of those which have low solubility in aqueous solution. An essential prerequisite for improved bioavailability of the problematic medicament is thus also provided.

For the preparation of the preparations according to the invention, a magnesium hydroxide carbonate having a specific particle morphology is particularly suitable, in particular the magnesium hydroxide carbonate described in WO 2011/095269 A1. It is distinguished by a suitable particle morphology together with a particularly large BET surface area and a high pore volume.

The magnesium hydroxide carbonate described here is, owing to its porous structure, readily soluble in an acidic and aqueous environment, such as gastric juice, and releases CO₂ gas. Depending on the size of a tablet produced from this magnesium hydroxide carbonate, this magnesium hydroxide carbonate can be employed as excipient material or filler in medicament preparations which disintegrate rapidly after being taken orally or for the production of fizzy drinks comprising active ingredient.

The specific properties of the magnesium hydroxide carbonates described in WO 2011/095269 A1 provide the formulator in the pharmaceutical industry with the possibility of also bringing problematic active ingredients into a form with rapid release of active ingredient. Particularly interesting is the possibility of influencing the dissolution rate and the solubility (also as supersaturation) and consequently influencing the bioavailability of low-solubility and/or poorly absorbable APIs. It is particularly advantageous that magnesium hydroxide carbonate as excipient is a substance which is listed in all pharmacopoeias, meaning that problems likewise cannot arise during registration for approval of the medicaments.

However, this principle may be advantageous not only in pharmacy, but also in other applications which are confronted with corresponding active-ingredient release problems, such as, for example, in analytical test systems, diagnostics, for the release of agricultural active ingredients, in veterinary medicine or in body implants.

For the preparation of the preparations according to the invention, a low-solubility API, such as, for example, fenofibrate, is dissolved fully in a suitable solvent. For the preparation of this solution, a solvent or solvent mixture is employed in which magnesium hydroxide carbonate is insoluble or has only very low solubility.

This solution is intimately mixed with a magnesium hydroxide carbonate having a particularly large BET surface area and a large pore volume. The solvent is then removed, for example by stripping off in vacuo, if necessary with gentle warming, or by freeze-drying. The resultant active ingredient/magnesium hydroxide carbonate conglomerate has, as further investigations have shown, improved in-vitro release behaviour, in particular also if it is processed further to give formulations in tablet form.

This type of formulation of low-solubility active ingredients is particularly advantageous if it is necessary to prepare preparations of the active ingredient with particularly low dosages. The strong bond between the active ingredient and the surface of the excipient material produces a free-flowing powder which, if desired, can be compressed to give tablets in which a constant active-ingredient concentration can be reliably guaranteed. However, the same also applies to active ingredients which are to be administered in a low-dose and in powder form. Since the active ingredient is strongly bonded to the excipient, separation of pulverulent excipient and active ingredient cannot occur.

Surprisingly, however, it has also been found that the advantageous properties of adsorption onto the porous excipient are retained if the low-solubility active ingredient is applied to the porous magnesium hydroxide carbonate as excipient from solution in relatively high concentration. Even under these conditions, the active ingredient has improved release of active ingredient, although the increased concentration in the solution during removal of the solvent used means that it would have been thought that the original physical and chemical properties of the low-solubility active ingredient predominate and prevail.

In particular in formulations in which the active ingredient has to be administered in very low doses, uniform distribution of the active ingredient is advantageous, since the strong bond to the excipient means that variations in the weight of the formulation result in smaller variations in the dosage than if the functional component were present in the form of separate particles.

Dosage forms are taken to mean all forms which are suitable for use as medicaments, in particular for oral administration, and as food supplements, but also cosmetics, agrochemicals, such as herbicides or fungicides, reagents, diagnostic products and animal feeds, and also as dyes, dietary minerals or catalysts. These include, for example, tablets in any shape, capsules, pellets or granules and powder mixtures.

Although the magnesium hydroxide carbonate employed in accordance with the invention is directly compressible, adjuvants may be present in accordance with the invention in the solid formulations comprising active ingredient, besides the active ingredient and the porous magnesium hydroxide carbonate as excipient. These may be, inter alia, flavour improvers, tableting assistants, such as glidants and lubricants, and the like. Possible additives are, for example, thermoplastic polymers, lipids, sugar alcohols, sugar alcohol derivatives, solubilisers, glidants and lubricants and others.

Suitable thermoplastic polymers are, for example, polyvinyl pyrrolidone (PVP), copolymers of N-vinylpyrrolidone and vinyl acetate or vinyl propionate, copolymers of vinyl acetate and crotonic acid, partially hydrolysed polyvinyl acetate, polyvinyl alcohol, polyhydroxyalkyl acrylates, polyhydroxyalkyl methacrylates, polyacrylates and polymethacrylates (Eudragit products), copolymers of methyl methacrylate and acrylic acid, polyethylene glycols, alkylcelluloses, in particular methylcellulose and ethylcellulose, hydroxyalkylcelluloses, in particular hydroxypropylcellulose (H PC), hydroxyalkylalkylcelluloses, in particular hydroxypropylmethylcellulose (HPMC), cellulose esters, such as cellulose phthalates, in particular cellulose acetate phthalate, hydroxypropylmethylcellulose phthalate and hydroxypropylmethylcellulose acetate succinate (HPMCAS). Thermoplastic polymers of this type are known to the person skilled in the art. He will be able to choose between the thermoplastic polymers which are commercially available for this purpose, depending on the desired properties of the tablets to be produced.

However, low-molecular-weight substances may also be present as additional excipients and fillers in the formulations comprising active ingredient. These can be sugars, such as sucrose, glucose, maltose, xylose, fructose, ribose, arabinose, galactose, trehalose, but also sugar alcohols. Suitable sugar alcohols are sorbitol, xylitol, mannitol, maltitol; a suitable sugar alcohol derivative is also isomaltitol. Urea, nicotinamide, amino acids or cyclodextrins may also be suitable. These additives may be commercially available in various grades under various trade names.

Suitable lipids are fatty acids, such as stearic acid; fatty alcohols, such as cetyl or stearyl alcohol; fats, such as animal or vegetable fats; waxes, such as carnauba wax; or mono- and/or diglycerides or phosphatides, in particular lecithin. The fats preferably have a melting point of at least 50° C. Preference is given to triglycerides of the C₁₂-, C₁₄-, C₁₆- and C₁₈-fatty acids.

In addition, conventional pharmaceutical-formulation adjuvants, whose total amount can be up to 20% by weight, preferably less than 10% by weight, in particular less than 5% by weight, based on the dosage form, can also be used. These include:

diluents or fillers, such as lactose, cellulose, silicates, phosphates or silicic acid;

lubricants, such as magnesium stearate and calcium stearate, sodium stearyl fumarate;

plasticisers;

dyes, such as azo dyes, organic or inorganic pigments or dyes of natural origin,

stabilisers, such as antioxidants, light stabilisers, hydroperoxide destroyers, free-radical scavengers, preservatives and stabilisers against microbial infestation;

aromas and fragrances;

anticaking agents;

disintegration-promoting adjuvants (disintegrants)

and retardation agents.

Active ingredients in the sense of the invention are taken to mean all substances having a desired physiological action on the human or animal body or plants. They are, in particular, active pharmaceutical ingredients. The amount of active ingredient per dose can vary within broad limits. It is generally selected so that it is sufficient in order to achieve the desired action. Combinations of active ingredients can also be employed. Active ingredients in the sense of the invention are in addition also vitamins and dietary minerals as well as peptide therapeutic agents and proteins.

Low-solubility substances in the sense of the invention are taken to mean substances whose saturation solubility at room temperature (20° C.) in at least one of the following media is less than 1% by weight: water, 0.1 molar aqueous hydrochloric acid, aqueous phosphate buffer pH 7.2, 0.9% by weight aqueous saline solution.

Low-solubility substances which come into consideration in accordance with the invention are a multiplicity of active ingredients and effect substances, in particular pharmaceutical or cosmetic active ingredients, active ingredients for food supplements or dietary products or food additives.

Low-solubility substances in the sense of the invention are, for example: piroxicam, clotrimazole, carbamazepine, 17-beta-estradiol, sulfathiazole, fenofibrate, benzocaine, lidocaine, dimetindene, biperiden, bisacodyl, clioquinol, droperidol, haloperidol, nifedipine, nitrendipine, tetracycline, phenytoin, glafenine, floctafenine, indometacin, ketoprofen, ibuprofen, dipyridamole, mefenaminic acid, amiodarone, felodipine, itraconazole, ketoconazole, danazole, furosemide, tolbutamide, ritonavir, lopinavir, naproxen, spironolactone, propafenone, progesterone, paclitaxel, docetaxel, theophylline, hydrocortisone, beta-carotene, vitamin A, tocopherol acetate, riboflavin, vitamin Q 10, vitamin D, vitamin K, disulfiram, nimodipine, chlorothiazide, chlorpropamide, dicoumarol, chloramphenicol, digoxin, lonidamine, pizotifen, atovaquone, amprenavir, bexarotene, calcitrol, clofazimine, doxercalciferol, dronabinol, durasteride, etoposide, loratadine, risperidone, saquinavir, sirolimus, valproic acid, amphotericin, alprostadil, carmustine, chlordiazepoxide, fenoldopam, melphalan, methocarbamol, oxytetracycline, docetaxel, fulvestrant, propofol, voriconazole, ziprasidone, leuprolide acetate, viadur, valrubicin, tramadol, celecoxib, etodolac, refocoxib, oxaprozin, leflunomide, diclofenac, nabumetone, flurbiprofen, tetrahydrocannabinol, capsaicin, ketorolac, albendazole, ivermectin, amiodarone, zileuton, zafirlukast, albuterol, montelukast, azithromycin, ciprofloxacin, clarithromycin, dirithromycin, rifabutin, rifapentin, trovafloxacin, baclofen, ritanovir, saquinavir, nelfinavir, efavirenz, dicoumarol, tirofiban, cilostazol, ticlidopin, clopidrogel, oprevelkin, paroxetine, sertraline, venlafaxine, bupropion, clomipramine, miglitol, repaglinide, glimepiride, pioglitazone, rosiglitazone, troglitazone, glyburide, glipizide, glibenclamide, fosphenytion, tiagabine, topiramate, lamotrigine, vigabatrin, amphotericin B, butenafine, terbinafine, itraconazole, flucanazole, miconazole, ketoconazole, metronidazole, griseofulvin, nitrofurantoin, lisinopril, benezepril, nifedipine, nisolidipine, telmisartan, irbesartan, eposartan, valsartan, candesartan, minoxidil, terazosin, halofantrine, mefloquine, dihydroergotamine, ergotamine, frovatriptan, pizotifen, sumatriptan, zolmitriptan, naratriptan, rizatriptan, aminoglutethimide, busulfan, cyclosporin, mitoxantrone, irinotecan, etoposide, teniposide, paclitaxel, tacrolimus, sirolimus, tamoxifen, camptothecan, topotecan, nilutamide, bicalutamide, toremifene, atovaquone, metronidazole, furazolidone, paricalcitol, benzonatate, midazolam, zolpidem, gabapentin, zopiclone, digoxin, beclometasone, budesonide, betamethasone, prednisolone, cisapride, cimetidine, loperamide, famotidine, lansoprazole, rabeprazole, nizatidine, omeprazole, cetirizine, cinnarizine, dexchlorpheniramine, loratadine, clemastine, fexofenadine, chlorpheniramine, acitretin, tazaroten, calciprotein, calcitriol, Targretin, isotretinoin, tretinoin, calcifediol, fenofibrate, probucol, gemfibrozil, cerivistatin, pravastatin, simvastatin, fluvastatin, atorvastatin, tizanidine, dantrolene, isosorbide dinitrate, dihydrotachysterol, essential fatty acids, codeine, fentanyl, methadone, nalbuphine, pentazocine, clomiphene, danazol, dihydroepiandrosterone, medroxyprogesterone, progesterone, rimexolone, megestrol acetate, oestradiol, finasteride, mefepristone, L-thyroxine, tamsulosin, methoxsalen, tacrine, donepezil, raloxifene, verteporfin, sibutramine, pyridotigmine, and isomers, derivatives, salts or mixtures thereof or other active ingredients which satisfy the above-mentioned definition.

If the release of fenofibrate as model active ingredient from a capsule is compared in release experiments, where the active ingredient is adsorbed from an acetone solution onto a magnesium hydroxide carbonate according to the invention (sample A) (Example 4), it is observed that this formulation results in considerably improved release of the active ingredient compared with formulations consisting of a mechanical mixture of the active ingredient and the magnesium hydroxide carbonate. In addition, release from this formulation is also more favourable than from corresponding formulations in which the magnesium hydroxide carbonate has been prepared in a different way to that described in WO 2011/095269 A1 and accordingly has lower porosity and a smaller surface area (Examples 5 and 6, fenofibrate adsorbed onto magnesium hydroxide carbonate samples B and C).

In summary, the experiments carried out show that the adsorption of a low-solubility active ingredient from a solution onto magnesium hydroxide carbonate as excipient is superior to a simple mechanical mixture of the components with respect to release rate and release amount of the active ingredient.

Surprisingly, however, a sample of magnesium hydroxide carbonate loaded with fenofibrate (from a solution) exhibits significantly improved behaviour with respect to the rate and extent of active-ingredient release (sample A in Example 4, magnesium hydroxide carbonate prepared by the process described in WO 2011/095269 A1). This outstanding release behaviour of sample A (Example 4) is found both in the release medium to which 1% of SDS has been added and also to which 0.5% of SDS has been added. The magnesium hydroxide carbonate of sample A differs from the other magnesium hydroxide carbonate grades investigated (samples B and C), in particular, in the BET surface area. The magnesium hydroxide carbonate of sample B exhibits a BET surface area of only about 11.5 m²/g, that of sample C does at least have a BET surface area of about 31.6 m²/g, but the magnesium hydroxide carbonate according to the invention of sample A has a BET surface area of 44.4 m²/g. The 31.6 m²/g BET surface area of sample C is apparently not yet sufficient for a significant improvement in the release of the active ingredient.

The data show that formulations of this type have faster and more comprehensive active-ingredient release than comparable mechanical mixtures of the components.

The present description enables the person skilled in the art to apply the invention comprehensively. Even without further comments, it is therefore assumed that a person skilled in the art will be able to utilise the above description in the broadest scope.

If anything is unclear, it goes without saying that the publications and patent literature cited should be consulted. Accordingly, these documents are regarded as part of the disclosure content of the present description. This applies, in particular, to the disclosure content of the application WO 2011/095269, in which the preparation of the magnesium hydroxide carbonate used is described and which is thus part of the disclosure content of the present invention.

For better understanding of the invention and in order to illustrate it, examples are given below which are within the scope of protection of the present invention. These examples also serve to illustrate possible variants. Owing to the general validity of the inventive principle described, however, the examples are not suitable for reducing the scope of protection of the present application to these alone.

Furthermore, it goes without saying to the person skilled in the art that, both in the examples given and also in the remainder of the description, the component amounts present in the compositions always only add up to 100% by weight, mol-% or % by vol., based on the composition as a whole, and cannot exceed this, even if higher values could arise from the per cent ranges indicated. Unless indicated otherwise, % data are thus regarded as % by weight, mol-% or % by vol.

The temperatures given in the examples and the description as well as in the claims are always in ° C.

### EXAMPLES

In order to carry out the following examples, the following materials, equipment and measurement methods were used:

### Equipment and Methods for the Characterisation of the Substance Properties

1. Bulk density: in accordance with DIN EN ISO 60: 1999 (German version)
result given in “g/ml”

2. Tapped density: in accordance with DIN EN ISO 787-11: 1995 (German version)
result given in “g/ml”

3. Surface area determined in accordance with BET: evaluation and procedure in accordance with the literature “BET Surface Area by Nitrogen Absorption” by S. Brunauer et al. (Journal of American Chemical Society, 60, 9, 1983) instrument: ASAP 2420 Micromeritics Instrument Corporation (USA); nitrogen; sample weight: about 3.0000 g; heating: 50° C. (5 h); heating rate 3 K/min; quoting of the arithmetic mean from three determinations

4. Particle size determination via laser diffraction with dry dispersal: Master-sizer 2000 with Scirocco 2000 dispersion unit (Malvern Instruments Ltd. UK), determinations at a counterpressure of 1 and 2 bar; Fraunhofer evaluation; dispersant RI: 1.000, obscuration limits: 0.0-10.0%, tray type: general purpose, background time: 7500 msec, measurement time: 7500 msec, procedure in accordance with ISO 13320-1 and the information in the technical manual and specifications from the instrument manufacturer; result given in % by vol.

5. Particle size determination via laser diffraction with wet dispersal: Master-sizer 2000 with Hydro 2000S wet-dispersion unit (Malvern Instruments Ltd., UK); dispersion medium deionised water; dispersant RI: 1.330; pump speed: 2000 rpm; stirrer speed: 2000 rpm; ultrasonic duration: 1 sec; ultrasonic level: 100%; tray type: general purpose; background time: 7500 msec; measurement time: 7500 msec; obscuration limits: 10.0-20.0%;

procedure in accordance with ISO 13320-1 and in accordance with the information in the technical manual and specifications from the instrument manufacturer; result given in % by vol.

6. Particle size determination by dry sieving via a sieve tower: Retsch AS 200 control, Retsch (Germany); amount of substance: about 110.00 g; sieving time: 30 minutes; amplitude intensity: 1 mm; interval: 5 seconds; analytical sieve with metal-wire fabric in accordance with DIN ISO 3310; mesh widths (in μm): 710, 600, 500, 400, 355, 300, 250, 200, 150, 100, 75, 50, 32; amount distribution per sieve fraction indicated in the tables as “% by weight of the sample weight”:

7. Release testing: The samples filled into a hard capsule are measured in an in-vitro release apparatus from ERWEKA (Heusenstamm, Germany) using the “Apparatus 2 (Paddle Apparatus)” described in USP35 under <711> and under the conditions described therein for “immediate-release dosage forms” (USP =United States Pharmacopoeia). In order that the capsules do not float, they are weighed down with a so-called “sinker” (see USP 35 under <711> Apparatus 2). The sample is drawn automatically via a hose-pump system with subsequent measurement in a Lambda 35 photometer, Perkin Elmer (USA) in a through-flow cell. The averaged values are obtained from 6 measurements.

### Measurement Apparatuses and Measurement Parameters

ERWEKA DT70 release apparatus fitted with Apparatus 2 (paddle apparatus in accordance with USP 35)

temperature: 37° C.+/−0.5° C.

rotary speed of the paddle: 75 rpm

volume of release medium per measurement vessel: 1000 ml

sample weight per capsule: 500 mg of substance

total run time of the measurement: 120 min. (with sample drawing after 5, 10, 15, 20, 25, 30, 45, 60, 75, 90 and 120 min.)

hose pump for sample drawing: Ismatec IPC, model ISM 931; App. No. 12369-00031

Lambda 35 photometer (Perkin Elmer)

measurement at 290 nm in a 2 mm through-flow measurement cell

evaluation via Dissolution Lab software version 1.1., Perkin Elmer Inc. (USA)

composition (in % by weight) of the release medium comprising 0.5% of SDS:
sodium chloride 0.2%
hydrochloric acid 1M 8.1%
SDS (sodium dodecylsulfate) 0.5%
deionised water 91.2%

composition (in % by weight) of the release medium comprising 1% of SDS:
sodium chloride 0.2%
hydrochloric acid 1M 8.1%
SDS (sodium dodecylsulfate) 1.0%
deionised water 90.7%

8. Assay of fenofibrate in the mechanical mixtures or in the fenofibrate/magnesium hydroxide carbonate conglomerates by HPLC via a LiChrospher 100 RP-18e 125-4 column Art. No. 150828 Merck, Darmstadt (Germany); pre-column: LiChrospher 100 RP-18e 4-4 Art. No. 150962; eluent: 69.93% by vol. of acetonitrile Art. No. 100030 Merck KGaA, Darmstadt, 29.97% by vol. of water LiChrosolv Art. No. 115333 Merck KGaA, Darmstadt and 0.10% by vol. of trifluoroacetic acid Art. No. 108262 Merck KGaA, Darmstadt (Germany), pH about1.5; flow rate: 2 ml/min.; pressure: about 65 bar; temperature: 50° C.; detection: 286 nm.

### Characterisation of the Materials Used

### Magnesium Hydroxide Carbonates Used

Sample A: Parteck Mg DC magnesium hydroxide carbonate heavy Ph Eur., BP, USP, E 504, Merck KGaA, Darmstadt (Germany), Art. No. 102440, batch: K0076840

Sample B: NutriMag MC DC magnesium carbonate heavy, pharm., gran. in purity BP, USP, Ph Eur.; CALMAGS GmbH, Lüneburg (Germany); batch: 308075060

Sample C: Pharmagnesia MC type A granules magnesium carbonate heavy Ph Eur., E 504, Lehmann & Voss & Co, Hamburg (Germany), batch: 0805-089, supplied batch: 4500068169

Additional characterisation of samples A, B and C with respect to bulk density, tapped density, BET surface area, BET pore volume, particle distribution via laser diffraction with wet dispersal (in water) and via tower sieving:

TABLE 1
Bulk density, tapped density, BET surface area, BET pore volume
(details of the measurement methods see under Methods)

| | Bulk | Tapped | BET | BET |
|---|---|---|---|---|
| | density | density | surface area | pore volume |
| Sample | (g/ml) | (g/ml) | (m²/g) | (cm³/g) |
| Sample A | 0.53 | 0.75 | 44.4 | 0.20 |
| Sample B | 0.63 | 0.77 | 11.5 | 0.08 |
| Sample C | 0.59 | 0.74 | 31.6 | 0.20 |

TABLE 2
Particle distribution determined via laser
diffraction with wet dispersal in water
Results given in μm (details of the
measurement method see under Methods)

| Sample | D(5) | D(10) | D(20) | D(25) | D(30) | D(50) |
|---|---|---|---|---|---|---|
| Sample A | 2.26 | 5.62 | 11.87 | 14.10 | 16.11 | 23.76 |
| Sample B | 1.24 | 2.02 | 4.37 | 6.01 | 7.84 | 15.96 |
| Sample C | 1.28 | 1.77 | 2.75 | 3.33 | 4.03 | 9.85 |

| Sample | D(75) | D(90) | D(95) | D(99) | D(100) |
|---|---|---|---|---|---|
| Sample A | 36.09 | 50.07 | 59.41 | 75.81 | 93.54 |
| Sample B | 31.37 | 67.61 | 197.37 | 455.59 | 684.57 |
| Sample C | 49.38 | 111.07 | 161.95 | 271.95 | 387.98 |

TABLE 3
Particle distribution determined via tower sieving
Results given in % by weight (details of the measurement method see under Methods)

| Sample | <32 μm | 32-50 μm | 50-75 μm | 75-100 μm | 100-150 μm | 150-200 μm | 200-250 μm |
|---|---|---|---|---|---|---|---|
| Sample A | 12.2 | 53.4 | 27.2 | 6.1 | 0.2 | 0.2 | 0.1 |
| Sample B | 0.1 | 0.3 | 0.4 | 1.1 | 5.2 | 10.9 | 12.4 |
| Sample C | 0.0 | 0.0 | 0.2 | 0.9 | 3.7 | 12.8 | 16.5 |

| Sample | 250-300 μm | 300-355 μm | 355-400 μm | 400-500 μm | 500-600 μm | 600-710 μm | >710 μm |
|---|---|---|---|---|---|---|---|
| Sample A | 0.1 | 0.1 | 0.1 | 0.0 | 0.1 | 0.2 | 0.0 |
| Sample B | 11.8 | 12.5 | 7.5 | 21.1 | 15.3 | 1.4 | 0.0 |
| Sample C | 17.8 | 14.9 | 13.0 | 17.8 | 2.4 | 0.0 | 0.0 |

### Model Active Ingredient Fenofibrate Used

Fenofibrate >=99% powder, Sigma Aldrich, Buchs (Switzerland), Pcode 101050968, F6020, LOT BCBG3328V

TABLE 4
Particle distribution determined via laser diffraction with dry dispersal
(various pressure conditions)
Results given in μm (details of the measurement method see under Methods)

| | Pressure | | | | | | | | | |
|---|---|---|---|---|---|---|---|---|---|---|
| | 1 bar | | | | | 2 bar | | | | |
| | Sample | | | | | | | | | |
| | D(10) | D(25) | D(50) | D(75) | D(90) | D(10) | D(25) | D(50) | D(75) | D(90) |
| Fenofibrate | 15.36 | 40.96 | 102.00 | 198.00 | 309.03 | 1.52 | 4.62 | 10.10 | 23.47 | 72.99 |
| powder | | | | | | | | | | |

### Solvent Acetone Used for Dissolving the Fenofibrate

Acetone suitable for use as excipient EMPROVE exp Ph Eur., BP, NF, Merck KGaA, Darmstadt (Germany), Art. No.: 100013, batch: K42947013

### Chemicals Used for the Preparation of the Release Media

Sodium dodecylsulfate, Ph Eur., Merck KGaA, Darmstadt (Germany), Art. No.: 817034, batch: K42572034 (“SDS” is used as abbreviation for sodium dodecylsulfate in the text and in the tables)

Sodium chloride suitable for use as excipient EMPROVE exp PH Eur., BP, USP, Merck KGaA, Darmstadt (Germany), Art. No.: 106400, batch: K93205800

Hydrochloric acid c(HCl)=1 mol/l (1N) TitriPUR, Merck KGaA, Darmstadt (Germany); Art. No.: 109057, batch: HC247274

Deionised water (abbreviation: “DI water”)

### Hard Gelatine Capsules Used

Capsugel Gr.00, product CD: X01 E0009, Capsugel Order No. 2159658-1-1, batch No. 33045581

### Magnesium Hydroxide Carbonate for Release of Active Ingredient

Aim: The aim of the experiments is to investigate differences in the in-vitro release behaviour of low-solubility active ingredients, such as, for example, fenofibrate, from formulations based on various types of magnesium hydroxide carbonate. To this end, firstly three different free-flowing and directly compressible magnesium hydroxide carbonates (in particular having different BET surface areas and pore volumes) are compared along with different incorporation methods of the active ingredient onto the excipient:

a) simple mechanical mixtures of active ingredient and excipient compared with

b) active ingredient adsorbed onto the excipient from solution.

### Experimental Results

### A) Examples 1-3

### Preparation of Mechanical Mixtures of Fenofibrate and Magnesium Hydroxide Carbonate Samples A-C and Measurement of the Release of Fenofibrate From Capsules Filled With These Mixtures

Principle:

In each case, 5.0 g of fenofibrate are added to 45.0 g of each of magnesium hydroxide carbonate samples A-C, the components are mixed, and 500 mg +/−2 mg of each of these mixtures are subsequently filled manually into hard capsules (in each case 12 capsules per mixture). In each case 6 of these capsules are tested for rate and extent of fenofibrate release in the Erweka paddle apparatus in both media (with 1% of SDS or 0.5% of SDS).

The measurements in the two release media with different amounts of SDS detergent serve for better discrimination of the different fenofibrate release behaviour based on the three different magnesium hydroxide carbonates.

Mixing Conditions:

Mixing vessel: 250 ml wide-neck clear-glass bottle, Order No. 215-1805, VW R, Germany

Mixer type: TurbulaT2A laboratory tumble mixer (Willy A. Bachofen, Switzerland)

TABLE 5

| | Weight of | Weight of |
|---|---|---|
| | samples A-C | fenofibrate |
| Example 1 | Sample A: 45.0 g | 5.0 g |
| Example 2 | Sample B: 45.0 g | 5.0 g |
| Example 3 | Sample C: 45.0 g | 5.0 g |

Mixing: after a mixing time of 5 min., the material is passed through a 1 mm hand sieve (diameter 20 mm; Retsch, Haan, Germany), and any lumps present are gently pressed through the sieve by hand using a plastic spatula: mixing is then carried out for a further 5 min.

The assay and homogeneity of the mixtures are determined: determination of the fenofibrate by HPLC from 6 preparation samples in each case where the rel. standard deviation must not be greater than 10%.

Then filling into hard capsules and measurement of the release behaviour.

TABLE 6
Results of fenofibrate release from the hard capsules in
the release medium with 1% of SDS/sodium dodecylsulfate
(Results given in % by weight of the amount of fenofibrate
released, based on an expected total amount of 50 mg of
fenofibrate/capsule; measurement of 6 capsules per example)

| Time (min.) | Min | Max | Mean |
|---|---|---|---|
| | Example 1 | | |
| 5 | 7.09 | 11.21 | 8.7 |
| 10 | 15.93 | 17.26 | 16.4 |
| 15 | 19.69 | 20.67 | 20.0 |
| 20 | 22.74 | 23.54 | 23.0 |
| 25 | 25.04 | 25.99 | 25.5 |
| 30 | 27.22 | 28.17 | 27.8 |
| 45 | 33.28 | 34.19 | 33.7 |
| 60 | 38.69 | 40.10 | 39.2 |
| 75 | 44.09 | 45.81 | 44.5 |
| 90 | 48.98 | 51.07 | 49.5 |
| 120 | 57.22 | 59.96 | 58.1 |
| | Example 2 | | |
| 5 | 3.72 | 6.08 | 5.0 |
| 10 | 7.42 | 11.38 | 10.4 |
| 15 | 12.28 | 15.42 | 14.4 |
| 20 | 16.11 | 18.72 | 17.5 |
| 25 | 19.44 | 21.83 | 20.5 |
| 30 | 21.86 | 24.43 | 23.1 |
| 45 | 29.31 | 32.83 | 31.4 |
| 60 | 36.12 | 41.37 | 39.2 |
| 75 | 42.20 | 49.38 | 46.2 |
| 90 | 47.55 | 56.23 | 52.4 |
| 120 | 56.17 | 67.38 | 62.4 |
| | Example 3 | | |
| 5 | 5.16 | 7.19 | 6.0 |
| 10 | 10.48 | 12.65 | 11.2 |
| 15 | 14.28 | 16.54 | 15.1 |
| 20 | 17.62 | 19.73 | 18.4 |
| 25 | 20.56 | 23.00 | 21.7 |
| 30 | 23.28 | 26.12 | 24.9 |
| 45 | 30.71 | 34.74 | 33.3 |
| 60 | 37.19 | 43.54 | 40.7 |
| 75 | 43.10 | 51.16 | 47.4 |
| 90 | 48.53 | 58.00 | 53.4 |
| 120 | 57.73 | 69.02 | 63.4 |

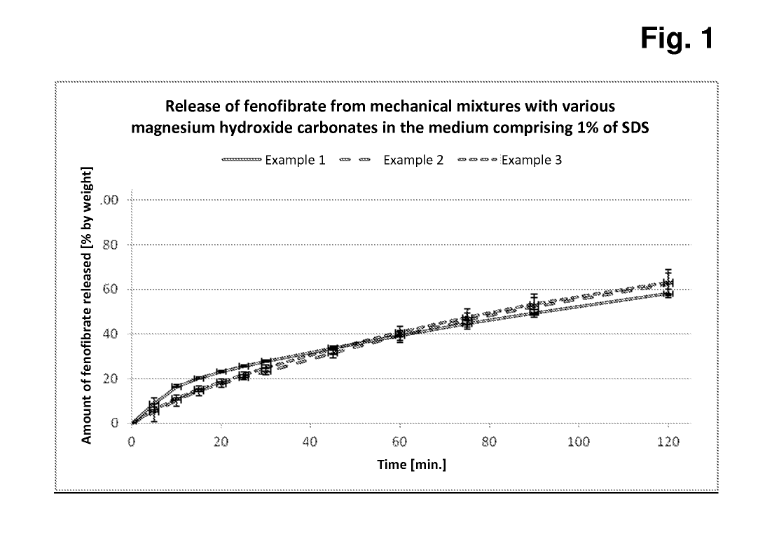

FIG. 1: The fenofibrate release profiles of the mechanical mixtures based on the three different magnesium hydroxide carbonates in the presence of 1% of SDS are shown (Examples 1,2 and 3); amount of fenofibrate released [% by weight] as a function of time [min].

TABLE 7
Results of fenofibrate release from the hard capsules in the release
medium with 0.5% of SDS (sodium dodecylsulfate)
(Results given in % by weight of the amount of fenofibrate released,
based on an expected total amount of 50 mg of fenofibrate/capsule;
measurement of 6 capsules per example)

| Time | Example 1 | | | Example 2 | | | Example 3 | | |
|---|---|---|---|---|---|---|---|---|---|
| (min.) | Min | Max | Mean | Min | Max | Mean | Min | Max | Mean |
| 5 | 5.07 | 9.54 | 7.5 | 4.55 | 5.39 | 5.0 | 6.24 | 19.27 | 9.3 |
| 10 | 12.22 | 13.66 | 12.8 | 7.86 | 9.02 | 8.3 | 10.95 | 24.62 | 13.9 |
| 15 | 14.19 | 15.27 | 14.7 | 9.83 | 11.72 | 10.6 | 14.06 | 28.26 | 17.0 |
| 20 | 15.63 | 16.66 | 16.1 | 11.37 | 13.55 | 12.5 | 16.24 | 30.71 | 19.2 |
| 25 | 16.89 | 17.88 | 17.4 | 12.71 | 15.41 | 14.1 | 17.60 | 30.08 | 20.1 |
| 30 | 18.18 | 19.04 | 18.6 | 14.09 | 17.23 | 15.7 | 19.32 | 31.93 | 22.0 |
| 45 | 21.94 | 22.48 | 22.2 | 18.10 | 22.19 | 20.1 | 23.77 | 36.33 | 26.5 |
| 60 | 25.20 | 26.26 | 25.6 | 22.23 | 27.18 | 24.4 | 28.17 | 40.31 | 30.7 |
| 75 | 28.36 | 29.88 | 28.9 | 26.51 | 31.82 | 28.6 | 32.39 | 44.09 | 34.8 |
| 90 | 31.51 | 33.27 | 32.1 | 30.67 | 35.99 | 32.6 | 36.38 | 48.13 | 38.8 |
| 120 | 37.54 | 39.90 | 38.2 | 37.21 | 43.45 | 39.6 | 43.48 | 55.40 | 46.2 |

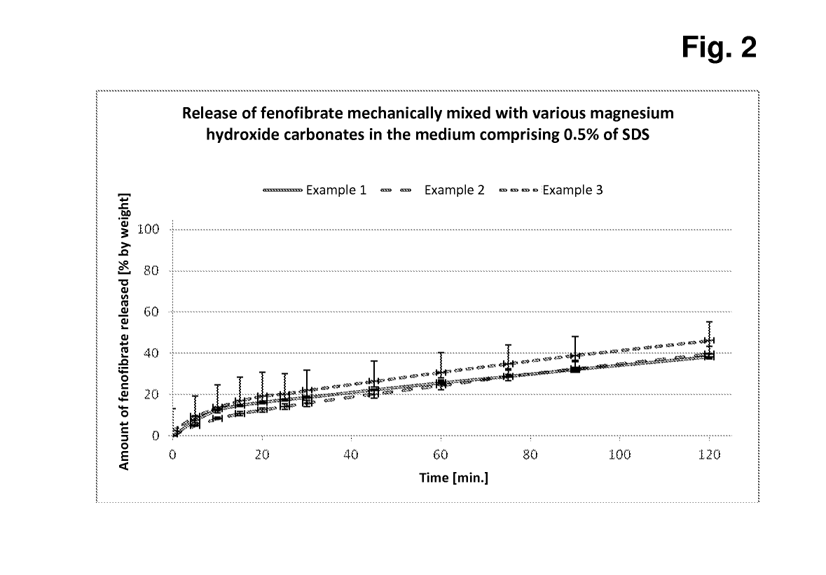

FIG. 2: The fenofibrate release profiles of the mechanical mixtures based on the three different magnesium hydroxide carbonates in the presence of 0.5% of SDS are shown (Examples 1, 2 and 3); amount of fenofibrate released [% by weight] as a function of time [min].

### Result of the Release Experiments of Fenofibrate From Physical Mixtures With Different Types of Magnesium Hydroxide Carbonate

The formulations of Examples 1-3 only differ slightly from one another in their release behaviour both in the presence of 1% of SDS and also in the presence of 0.5% of SDS. The extent of the release after 120 minutes is—as expected—slightly higher in the case of the larger amount of SDS in the release medium (1%) compared with the lower amount (0.5%). A discriminating influence of the magnesium hydroxide carbonates used is not evident.

### B) Examples 4-6

### Preparation of Magnesium Hydroxide Carbonate Samples A-C to Which Dissolved Fenofibrate Has Been Added, Removal of the Solvent and Measurement of the Release of Fenofibrate From Capsules Filled With These Fenofibrate/Magnesium Hydroxide Carbonate Conglomerates

Principle:

In each case, 50.0 g of fenofibrate (dissolved in acetone) are added to 450.0 g of each of magnesium hydroxide carbonate samples A-C, the components are mixed intimately, the solvent is removed, and 500 mg +/−2 mg of each of these conglomerates are subsequently filled manually into hard capsules (in each case 12 capsules per preparation). In each case 6 of these capsules are tested for rate and extent of fenofibrate release in the Erweka paddle apparatus in both media (with 1% of SDS or with 0.5% of SDS).

The measurements in the two release media with different amounts of SDS detergent serve for better discrimination of the different fenofibrate release behaviour based on the three different magnesium hydroxide carbonates.

Preparation:

In each case, 450.0 g of magnesium hydroxide carbonate samples A-C are initially introduced in a Hobart N-50 mixer with light-metal flat stirrer (Hobart Canada, North York, Ontario, Canada) and mixed for 2 min. The clear, colourless fenofibrate solution (50.0 g of fenofibrate dissolved in 250 g of acetone to give a clear solution) is then added as quickly as possible within about 10 sec.

TABLE 8

| | Weight of | Weight of |
|---|---|---|
| | samples A-C | fenofibrate |
| Example 4 | Sample A: 450.0 g | 50.0 g |
| Example 5 | Sample B: 450.0 g | 50.0 g |
| Example 6 | Sample C: 450.0 g | 50.0 g |

Mixing is carried out for a further 2 min.—cover the mixing vessel during this operation in order to prevent evaporation of the acetone—switch off the mixer, cover the mixing vessel with a glass and allow to stand for 15 minutes. The acetone is then removed in a Heraeus VT 5050 EK vacuum drying cabinet (23 hours at 50° C.; vacuum: about 180 mbar), the product is passed through a 1 mm sieve, and an assay and homogeneity test is carried out: determination of the fenofibrate by HPLC from 5 preparation samples in each case where the rel. standard deviation must not be greater than 10%.

Then filling into hard capsules and measurement of the release behaviour.

TABLE 9
Results of fenofibrate release from the hard capsules in the release
medium with 1% of SDS (sodium dodecylsulfate)
(Results given in % by weight of the amount of fenofibrate released, based
on an expected total amount of 50 mg of fenofibrate/capsule; measurement
of 6 capsules per example)

| | Example 4 | | | Example 5 | | | Example 6 | | |
|---|---|---|---|---|---|---|---|---|---|
| Time (min.) | Min | Max | Mean | Min | Max | Mean | Min | Max | Mean |
| 5 | 32.58 | 49.71 | 42.0 | 14.70 | 19.69 | 17.1 | 15.18 | 23.26 | 20.2 |
| 10 | 62.89 | 69.66 | 66.6 | 28.47 | 34.86 | 32.6 | 28.96 | 39.17 | 35.6 |
| 15 | 72.49 | 78.30 | 76.2 | 38.08 | 45.71 | 43.6 | 39.37 | 49.60 | 46.5 |
| 20 | 77.43 | 84.05 | 81.8 | 44.98 | 53.22 | 51.1 | 46.10 | 56.28 | 53.5 |
| 25 | 81.01 | 87.96 | 85.5 | 49.82 | 58.52 | 56.4 | 50.76 | 60.85 | 58.5 |
| 30 | 83.78 | 90.70 | 88.2 | 53.78 | 62.56 | 60.3 | 53.99 | 64.63 | 62.2 |
| 45 | 89.76 | 94.99 | 92.8 | 64.48 | 71.38 | 69.1 | 61.78 | 72.17 | 69.9 |
| 60 | 92.64 | 97.31 | 95.5 | 72.55 | 77.08 | 75.3 | 69.40 | 77.07 | 75.4 |
| 75 | 94.15 | 98.91 | 97.2 | 78.20 | 81.53 | 79.9 | 76.40 | 80.99 | 79.6 |
| 90 | 95.51 | 100.03 | 98.3 | 81.75 | 84.99 | 83.4 | 80.72 | 83.93 | 82.6 |
| 120 | 97.21 | 101.51 | 99.7 | 86.57 | 89.56 | 87.9 | 84.37 | 87.94 | 86.5 |

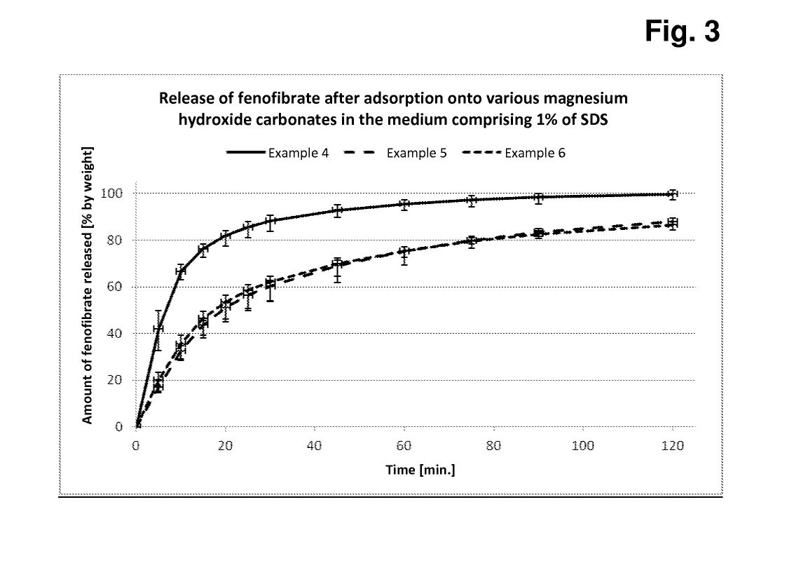

FIG. 3: The release profiles of fenofibrate [% by weight] as a function of time [min] after adsorption onto various magnesium hydroxide carbonates in the medium with 1% of SDS are shown (Examples 4, 5 and 6).

TABLE 10
Results of fenofibrate release from the hard capsules in the
release medium with 0.5% of SDS (sodium dodecylsulfate)
(Results given in % by weight of the amount of fenofibrate released, based
on an expected total amount of 50 mg of fenofibrate/capsule; measurement
of 6 capsules per example)

| | Example 4 | | | Example 5 | | | Example 6 | | |
|---|---|---|---|---|---|---|---|---|---|
| Time (min.) | Min | Max | Mean | Min | Max | Mean | Min | Max | Mean |
| 5 | 32.08 | 48.37 | 40.8 | 14.65 | 17.05 | 15.71 | 13.89 | 17.57 | 16.0 |
| 10 | 46.51 | 67.42 | 59.1 | 23.49 | 27.99 | 26.75 | 25.91 | 31.05 | 28.1 |
| 15 | 59.20 | 76.48 | 67.8 | 30.28 | 36.40 | 34.79 | 34.20 | 39.60 | 36.8 |
| 20 | 66.23 | 81.33 | 73.0 | 35.40 | 42.43 | 40.59 | 38.53 | 45.05 | 42.7 |
| 25 | 70.76 | 85.27 | 76.7 | 39.09 | 46.58 | 44.73 | 41.46 | 49.21 | 47.0 |
| 30 | 73.90 | 87.03 | 79.2 | 41.92 | 49.52 | 47.81 | 43.82 | 52.36 | 50.0 |
| 45 | 79.44 | 92.99 | 84.6 | 47.83 | 56.15 | 54.29 | 49.46 | 58.56 | 56.3 |
| 60 | 82.99 | 96.82 | 87.9 | 52.16 | 60.92 | 58.93 | 55.72 | 63.06 | 61.3 |
| 75 | 85.17 | 97.77 | 90.4 | 56.19 | 64.73 | 62.70 | 60.69 | 66.66 | 65.2 |
| 90 | 86.73 | 101.05 | 92.5 | 59.83 | 67.95 | 65.89 | 64.91 | 69.45 | 68.3 |
| 120 | 88.99 | 105.90 | 95.7 | 66.83 | 73.10 | 71.28 | 70.51 | 73.80 | 72.9 |

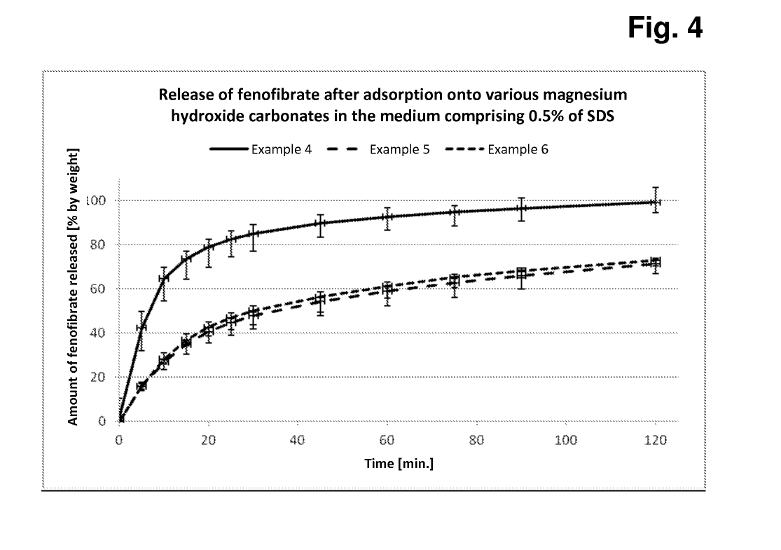

FIG. 4: The release profiles of fenofibrate [% by weight] as a function of time [min] after adsorption onto various magnesium hydroxide carbonates in the medium with 0.5% of SDS are shown (Examples 4, 5 and 6).

Result of the release experiments of fenofibrate adsorbed onto various grades of magnesium hydroxide carbonate: Both in the release medium with 1% of SDS and also with only 0.5% of SDS, there is significant differentiation in the release behaviour between the 3 examples 4-6. Fenofibrate adsorbed onto magnesium hydroxide carbonate sample A in accordance with Example 4 exhibits significantly faster and greater active-ingredient release, both in rate and also in extent (after 120 minutes), than fenofibrate adsorbed onto magnesium hydroxide carbonate samples B and C (Examples 5 and 6).

### C) Comparative Representation of the Different Release Behaviour of Fenofibrate From Mechanical Mixtures (Examples 1-3) and From Conglomerates

(obtained by adsorption of dissolved fenofibrate onto various magnesium hydroxide carbonates (Examples 4-6); release in a medium with addition of 1% of SDS)

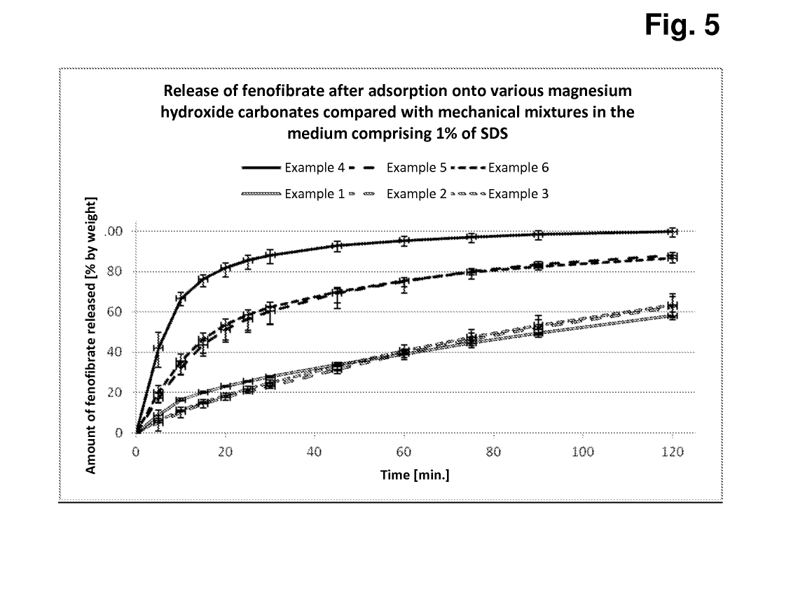

FIG. 5: The release profiles of fenofibrate after adsorption onto various magnesium hydroxide carbonates compared with the profiles of the mechanical mixtures in the presence of 1% of SDS are shown; Examples 4-6 compared with Examples 1-3.

Result: In the release medium with 1% of SDS as detergent, the fenofibrate formulation from Example 4 (fenofibrate adsorbed onto magnesium hydroxide carbonate sample A) exhibits both the fastest and also the most comprehensive release of the active ingredient in terms of amount. Examples 5 and 6 (fenofibrate adsorbed onto magnesium hydroxide carbonate samples B and C) show a fenofibrate release which is significantly slowed and also reduced in extent. By contrast, the mechanical mixtures are significantly less favourable in their fenofibrate release behaviour and also exhibit no discrimination between the 3 magnesium hydroxide carbonate samples used.

### D) Comparative Representation of the Different Release Behaviour of Fenofibrate From Mechanical Mixtures (Examples 1-3) and From Conglomerates

(obtained by adsorption of dissolved fenofibrate onto various magnesium hydroxide carbonates (Examples 4-6); release in a medium with addition of 0.5% of SDS)

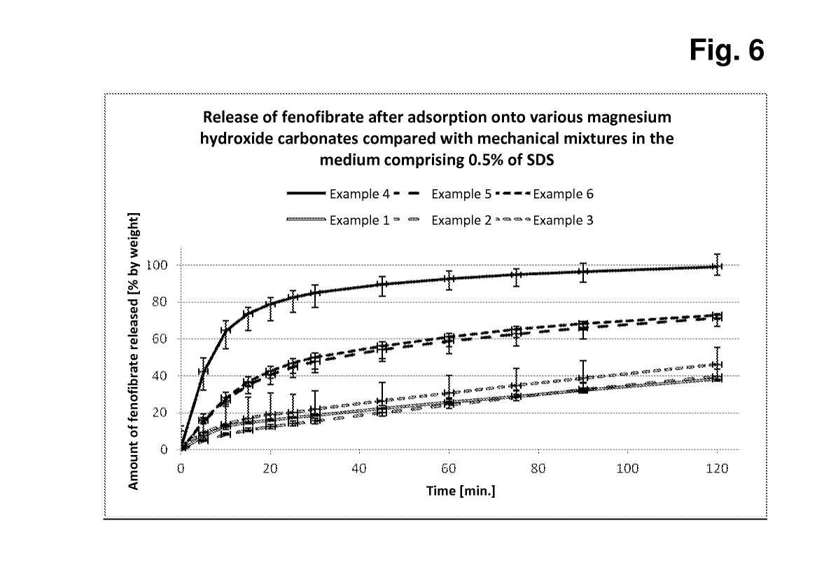

FIG. 6: The release profiles of fenofibrate after adsorption onto various magnesium hydroxide carbonates compared with the profiles of the mechanical mixtures in the presence of 0.5% of SDS are shown; Examples 4-6 compared with Examples 1-3.

Result: In the release medium with only 0.5% of SDS as detergent, the fenofibrate formulation from Example 4 (fenofibrate adsorbed onto magnesium hydroxide carbonate sample A) likewise exhibits both the fastest and also the most comprehensive release of the active ingredient in terms of amount. Examples 5 and 6 (fenofibrate adsorbed onto magnesium hydroxide carbonate samples B and C) show a fenofibrate release which is significantly slowed and also reduced in extent. By contrast, the mechanical mixtures are significantly less favourable in their fenofibrate release behaviour and also exhibit no discrimination between the 3 magnesium hydroxide carbonate samples used.

### E) Conclusion From the Experiments

1. The adsorption of fenofibrate from a solution onto the magnesium hydroxide carbonate excipients is superior to a simple mechanical mixture of the components with respect to release rate and release amount of the active ingredient.

2. Mechanical mixtures exhibit no release differentiation between the magnesium hydroxide carbonate grades tested.

3. Surprisingly, however, a sample of the magnesium hydroxide carbonates loaded with fenofibrate (from a solution) exhibits significantly improved behaviour with respect to the rate and extent of active-ingredient release (sample A in Example 4). This excellent release behaviour of sample A (Example 4) is found both in the release medium with 1% of SDS and also with 0.5% of SDS.

4. Sample A differs from samples B and C, in particular, in the BET surface area; sample C exhibits a BET surface area of only about 11.5 m²/g, sample B does at least exhibit a BET surface area of about 31.6m²/g and sample A exhibits a BET surface area of 44.4 m²/g. The BET surface area of sample C of 31.6m²/g is apparently not yet sufficient for a significant improvement in release.

The data show that formulations according to the invention have faster and more comprehensive active-ingredient release than comparable mechanical mixtures of the components. It is furthermore shown that selection of a magnesium hydroxide carbonate having a particularly large surface area and high pore volume enables an additional improvement to be achieved in the in-vitro release behaviour of an active ingredient which has low water solubility.

1. Formulations having accelerated active-ingredient release, comprising porous magnesium hydroxide carbonate having a large surface area as excipient material and at least one active ingredient.

2. Formulation according to claim 1, characterised in that it is a pharmaceutical formulation which comprises at least one pharmaceutical active ingredient.

3. Formulation according to claim 1, comprising at least one low-solubility active ingredient from BCS classes II and IV.

4. Formulations according to claim 1 obtainable by adsorption of the active ingredient or active ingredients from a solution onto the porous magnesium hydroxide carbonate as excipient material.

5. Formulations according to claim 1, comprising magnesium hydroxide carbonate having a BET surface area of at least 20 m²/g, preferably at least 30 m²/g and particularly preferably at least 40 m²/g.

6. Formulations according to claim 1, comprising porous magnesium hydroxide carbonate having a BET pore volume of at least 0.1 cm³/g as excipient material.

7. Process for the preparation of the formulations according to claim 1 characterised in that the active ingredient(s) is (are) dissolved in a suitable solvent or solvent mixture in which the porous magnesium hydroxide carbonate is insoluble, and the resultant solution is mixed intimately with the magnesium hydroxide carbonate, and the solvent or solvent mixture is removed again after the mixing.

8. Process according to claim 7, characterised in that the solution comprising active ingredient is brought into intimate contact at low temperature by spraying onto a porous magnesium hydroxide carbonate which is initially introduced in a fluidised bed, and is dried.

9. Process according to claim 7, characterised in that the porous magnesium hydroxide carbonate is suspended in the solution comprising active ingredient and is subsequently dried in a spray- or freeze-drying process.
